(19) United States
(12) Patent Application Publication
(10) Pub. No.: US 2009/0260615 A1
(43) Pub. Date: Oct. 22, 2009

(54) Warmer for Feet

(76) Inventor: Tim B. Kramer, Benton, PA (US)

Correspondence Address: Charles L. Riddle, Esq., 434 Lackawanna Ave, Suite 200, Scranton, PA, 19465, US

(21) Appl. No.: 12/424,936

(22) Filed: Apr. 16, 2009

Related U.S. Application Data
(60) Provisional application No. 61/045,964, filed on Apr. 18, 2008.

(51) Int. Cl. A61F 7/08

(52) U.S. Cl. 126/204

(57) ABSTRACT

An improved warmer for feet [1000] is described. The device [1000] allows a person [1] to insert both feet [7] into the warmer [1000]. The collective heat of both feet [7] causes air to circulate equalizing the temperature warming toes [9] which are most sensitive to hypothermia. A rigid member [1103] holds an open end [1101] open to allow insertion of the feet [7] of person [1] without the need for person [1] to use his hands. Alternatively, at least one elastic band [1113] causes open end [1101] to conform to the boots [5] reducing the leakage of heated air for inside of the warmer [1000]. Warmer [1000] is well suited for use during hunting from a tree stand where person 1 is motionless for long periods of time in the cold.

### CROSS REFERENCE TO RELATED APPLICATIONS

This patent application is related to, and claims priority from U.S. provisional Patent Application Ser. No. 61/045,964 filed Apr. 18, 2008 entitled “Warmer for Feet” by Tim B. Kramer. All content of this parent application is incorporated by reference as if set forth in its entirety herein.

### BACKGROUND OF THE INVENTION

1. Field of the Invention

The invention generally relates to an improved device for warming a user's feet and more particularly, for an improved device for warming both user's feet when stationary.

2. Discussion of Related Prior Art

During deer hunting and other outdoor activities, a person will spend long periods of time at one location and move very little. This is done so that they are not noticed by the animals.

These hunting seasons are typically in the cold weather. They usually spend a long period of time in the cold weather. They may be outside for periods of 6-8 hours. The longer one is out in the cold, the colder it feels.

The cold is also amplified by the fact that the person moves very little. Motion creates heat, and therefore less motion causes the person to feel colder.

For deer hunting, it is common to have a tree stand which attaches to a tree some distance from the ground. As one is elevated, there are typically fewer objects to block the wind.

The person stands on the tree stand. Typically, there is little extra room for other items on the stand besides the person's feet.

When a deer or other animal is in sight, the person may want to take a different stance to fire a rifle. If a blanket or other warming device is used, the person may not quickly release their feet to take action, causing a great disadvantage.

Also, one is typically a great distance out in the woods where there is no access to electricity. Also, the person cannot make a fire since it will alert the animals.

There are chemical hand warmers and foot warmers. These only last for a given period of time, are not reusable and end up as litter after being used.

Currently, there is a need for a device for efficiently warming a user's feet that is reusable and will fit in the areas where they are typically used, such as a tree stand.

### SUMMARY OF THE INVENTION

The present invention may be embodied as an improved warming device [1000] for warming toes [9] of feet [7] of a user [1] in boots [5] comprising:
a) base portion [1109] having two edges on either side for resting generally flat on a surface [3];
b) side portions [1105] connected to each edge of the base portion [1109],
c) an instep portion 1107 connected to each of the sides for creating a pocket with an open end [1101] allowing said user [1] to insert said feet [7] in boots [5] into open end [ 1101 ] of warming device [ 1000] resulting in improved warming of said toes [9] of user [1].

### OBJECTS OF THE INVENTION

It is an object of the present invention to reduce loss of the collective heat of both of a user's feet.

It is another object of the present invention to provide a covering having insulating air to keep a user's feet warm.

It is another object of the present invention to provide an improved heating device which may be put on and taken off of the user's feet without the need to use one's hands.

It is another object of the present invention to provide an improved feet heating device which will not fall down when used on a stationary tree stand.

It is another object of the present invention to provide an improved device for heating a user's feet which primarily heats the user's toes.

### BRIEF DESCRIPTION OF THE DRAWINGS

The advantages, nature and various additional features of the invention will appear more fully upon consideration of the illustrative embodiment of the invention which is schematically set forth in the drawings, in which:

FIG. 1 is a perspective view of one embodiment of an improved warmer according to the present invention.

FIG. 2 is a top plan view of the warmer of FIG. 1.

FIG. 3 is a side elevational view of the warmer of FIG. 1.

FIG. 4 is a side elevational view of another embodiment of an improved warmer according to the present invention.

### DETAILED DESCRIPTION OF THE DRAWINGS

While the invention is open to various modifications and alternative forms, specific embodiments thereof are shown by way of examples in the drawings and are described herein in detail. There is no intent to limit the invention to the particular forms disclosed.

### Overview

The present invention is an improved device for warming a person's feet when they are stationary for a period of time. It does not use chemical or electrical energy, and is reusable.

Also, it is known that a person's toes get colder before the remainder of one's feet. It is also known that mittens keep a person's hands much warmer than gloves of comparable thickness.

The idea is to use the concept of the mitten to keep both feet warm.

The design not only efficiently warms the person's feet, but allows the person to remove his/her feet quickly.

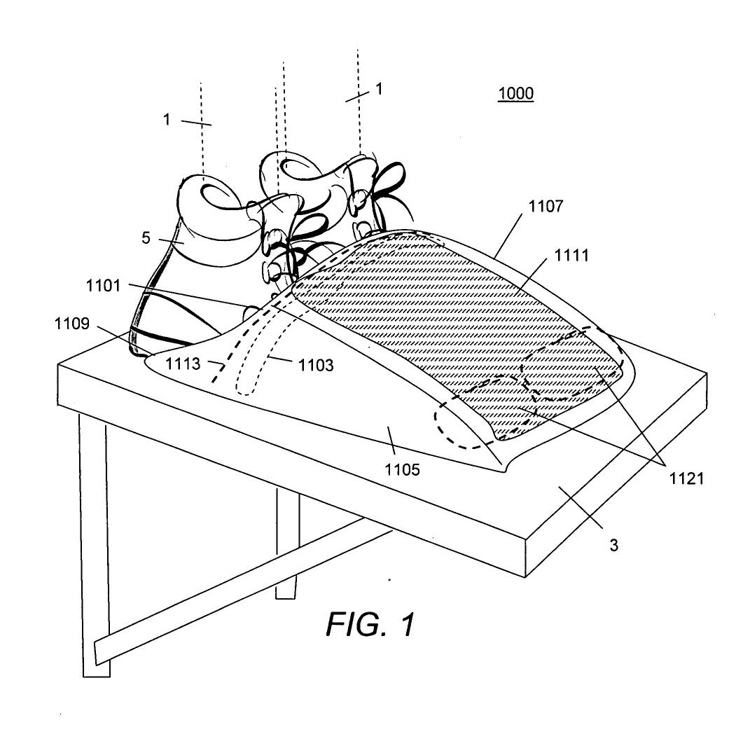

FIG. 1 is a perspective view of one embodiment of the improved device for heating a user's feet according of the present invention.

In this view it can be seen that a person is standing on a platform 3. The person 1 may be a hunter and the platform 3 may be part of a tree stand attached to a tree. Boots 5 of person 1 are shown inside of the warmer 1000.

Warmer 1000 has sides 1105, and an instep portion 1107 attached to the sides 1105. These are connected to minimize air leakage and maximize warmth. These are also made of a material which resists water, and is thermally insulated.

Alternatively, sides 1105, and instep portion 1107 may be constructed from a single piece of material.

Since a person's feet are exposed to vertical rain while standing, it is important to design warmer 1000 such that they allow rain to run off of instep portion 1107 keeping the feet of person 1 dry. This is important since most frostbite occurs in a person's toes.

Since the person will be stationary standing on the platform 3 almost motionless for a long period of time, it is possible for the person 1 to slip his feet inside of boots 5 into an open end 1101 of warmer 1000.

Warmer 1000 may optionally have elastic band 1113 sewn into the edge of open end 1101. The elastic band 1113 causes the open end 1101 to fit snugly against boots 5 of person 1.

Warmer 1000 may optionally have a semi-rigid member 1103 which encircles open end 1101. The semi-rigid member 1103 holds open end 1101 in an open position. This allows person 1 to slip boots 5 into open end 1101 without requiring the person 1 to use their hands. This is very convenient for its intended use since person 1 in a tree stand will probably be several feet high. Also, the person 1, if a hunter will be holding a loaded rifle and have to bend and balance to place his feet and boots 5 into any other type of warming device.

What further complicates the situation is that the person 1 will probably be using gloves. Removing the gloves and bending to place the feet into a device causes the potential of dropping items, such as gloves to the ground. It can become a tough matter to get down from the tree stand to retrieve the glove and situate the person 1 again. Therefore, a device which can be used without the use of the hands of person 1 is a great benefit in this situation.

As we had seen, the person 1 can put his feet into the warmer 1000 without the need of using the hands of person 1. The person 1 can also remove their feet from the device. Usually, if one were to use a blanket or other warming device, there is the problem of storage when not in use. Since person 1 is on a small platform, possibly perched in a tree, it is difficult to bend down, pick up and store without dropping these devices.

The present invention is designed to flatten when not in use and the person can stand on it without causing damage to the warmer 1000.

In one embodiment, the semi-rigid member 1103 is allowed to flatten. The person 1 then stands on an optional non-slip top covering 1111 on top of the flattened warmer 1000. This allows the entire platform to be used.

In an alternative embodiment, the semi-rigid member 1103 can have a pair of hinged joints allowing the sides 1105 to fold and instep 1107 to flatten against the base portion 1109.

Base portion 1109 may be made of a non-slip material which reduces slippage of warmer 1000 on platform 3.

Base portion 1109 and a portion of sides 1105 may also be made of a waterproof material which allows the person's 1 toes to remain dry when standing on wet ground.

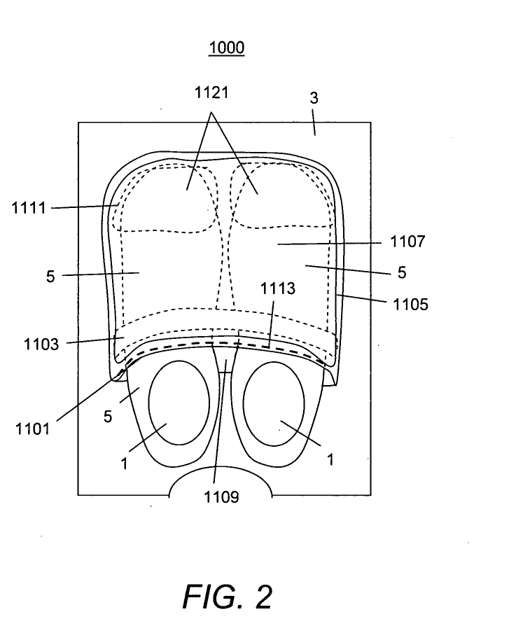

FIG. 2 is a top plan view of the warmer of FIG. 1. In this view one can easily see boots 5 inserted into open end 1101 of warmer 1000.

Sides 1105 are attached to base portion 1109 and to instep portion 1107 to create a continuous pocket into which boots 5 are inserted. Base portion 1109 is preferably attached to base 3 to prevent movement of the warmer 1000, making it easier to insert boots 5.

Rigid member 1103 is preferably used to hold open end 1101 to allow the person 1 to insert their feet and boots into warmer 1000 with the need to use their hands.

Elastic band 1113 is also preferably used in various embodiments and is shown in phantom to hold the edge of the open end 1101 against the boots 5 of person 1 to hold in warm air making it more efficient.

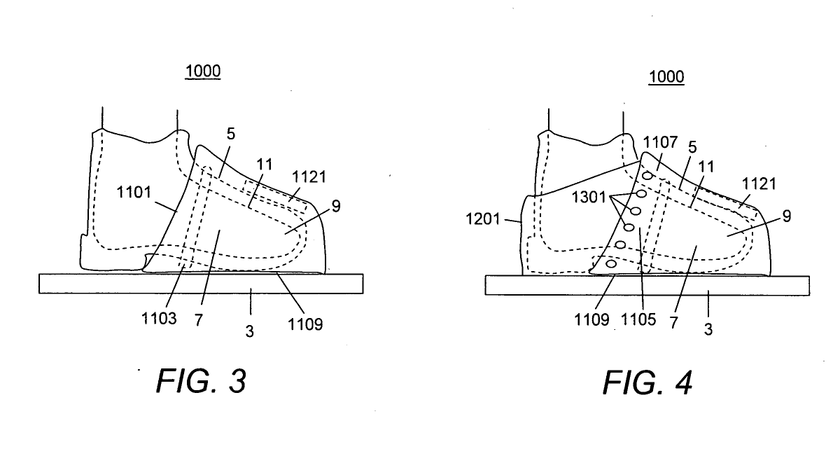

FIG. 3 is a side elevational view of the warmer of FIG. 1. Here the feet 7 and toes 9 of person 1 can be seen in boots 5 inside of warmer 1000. It can also be seen that attachment devices 1103 may be used to attach warmer 1000 to base 3. These may be any commonly known attachment device, such as snaps, clips or hook-and-loop attachment pads.

FIG. 4 is a side elevational view of another embodiment of an improved warmer according to the present invention. In this embodiment, a heel wrap portion 1201 wraps around the heel section and connects to the open end 1101, sides 1105, instep 1107 and/or base portion 1109. Again, any commonly known attachment device 1213 may be used, such as snaps, clips or hook-and-loop attachment pads.

The purpose of the heel wrap 1201 is to hold the boots 5 in warmer 1000, reduce air leakage out of the warmer 1000 increasing efficiency, and reducing direct heat loss from the ankles and heel of person 1 to the environment.

In an alternative embodiment, at least one pocket 1121 is built into the improved warmer 1000 as shown in FIGS. 1-4. This is designed to hold a chemical hand or foot warmer packet. These are typically chemically based and require oxygen to function. There is not a great deal of extra air with oxygen present in boots. Therefore, these packets tend to lose their efficiency as their access to oxygen is reduced.

Since the warmer 1000 has an open end 1101, there is enough of a supply of air to continuously fuel the improved warmer 1000.

When not in use, improved warmer may be rolled like a sleeping bag into a small package. Since this is intended for use outdoors, usually a long distance into the woods, it is very beneficial to have a device which is compact and lightweight. This reduced the size and weight of the equipment required to be carried.

Also, above it was described for use on a tree stand. This is a good use for the present invention. However, it will be useful anywhere it is cold and the user is committed to staying there for a period of time.

It is of course understood that departures can be made from the preferred embodiments of the invention by those of ordinary skill in the art without departing from the spirit and scope of the invention.

1. A warming device for warming toes of feet of a user in boots comprising:
a base portion having two edges on either side for resting generally flat on a surface,
side portions connected to each edge of the base portion, and
an instep portion connected to each of the sides for creating a pocket with an open end allowing said user to insert said feet in boots into open end of the warming device resulting in improved warming of said toes of the user.

2. The warming device of claim 1, the instep portion having a pitch to allow water to run off the instep portion.

3. The device of claim 2, the sides and instep portion formed as a single member or made from a single piece of material.

4. The device of claim 2, further including an elastic member formed as part of, sewn in, or attached to the open end of the warming device to cause the open end to fit snugly against the boots of the user.

5. The device of claim 2, further having a semi-rigid member formed as part of, sewn in, or attached on or near the open end of the warming device to hold open the open end and allow the user to slip boots into the open end without requiring the user to use a hand.

6. The device of claim 2, the side portions and the instep portion being collapsible to allow the user to stand on the warming device while said device is collapsed, and accomplish the same without damaging said device.

7. The device of claim 5, the semi-rigid member having at least one hingeable joint to allow the sides to fold and instep to flatten against the base portion.

8. The device of claim 1, the base portion comprising a non-slip material for reducing the warming device from slipping on the base.

9. The device of claim 7, the base portion having a mount for attaching the warming device to a platform prevent the warming device from moving relative to the platform to make inserting the boots into the pocket easier.

10. The device of claim 2, further including a heel wrap attached to the base portion, and/or the side portions, and/or the instep portion with an attachment device for covering portions of the boots that are not otherwise insertable into the open end.

11. The device of claim 10, the instep portion, and/or the side portions, and/or the base portion having a warmer member formed therein or attached thereto for holding at least one warmer packet.

12. A feet warming system for minimizing heat loss of feet of a user comprising:
a standing surface;
a pouch, the pouch having:
at least one pouch wall forming a periphery that defines an opening in the pouch,
a boot enveloping volume defined as the volume existing within the at least one wall and extending to the periphery, and
a base contacting area formed by the at least one wall and existing outside of the boot enveloping volume;
wherein the pouch is positioned on the standing surface with the base contacting area placed on the standing surface, and the user inserts the user's feet into the boot enveloping volume while the pouch is positioned on the standing surface.

13. A method of minimizing exposure of a user's feet to cold temperatures comprising the steps of:
providing or locating a standing surface;
providing a pouch, the pouch having:
at least one pouch wall forming a periphery that defines an opening in the pouch,
a dual boot enveloping volume defined as the volume existing within the at least one wall and extending to the periphery, and
a base contacting area formed by the at least one wall and existing outside of the dual boot enveloping volume;
placing the pouch on the standing surface with the base contacting surface oriented downward in contact with the standing surface;
inserting one or more feet into the boot enveloping volume of the pouch; and
standing or sitting with one or more feet inserted into the enveloping volume of the pouch.

14. The method of claim 13, further including the step of attaching the pouch to the standing surface to prevent the pouch from sliding.

15. The method of claim 13, the step of inserting one or more feet into the boot enveloping volume accomplished while carrying one or more items in the user's hands.

16. The method of claim 13, further including the step of covering with a heel wrap any remaining portion of the feet not within the boot enveloping volume.

17. The method of claim 15, further including the step of removing the one or more feet from the boot enveloping volume, collapsing the pocket, and standing on the pocket.

18. The method of claim 17, the step of removing the one or more feet from the boot enveloping volume accomplished while carrying one or more items in the user's hands.

19. The method of claim 13, wherein the step of providing or locating a standing surface includes placing a platform in a tree.

20. The method of claim 13, further including inserting a heat generating article into a pocket attached to or made part of the pouch, or inserting a heat generating into the boot enveloping volume.
